(19) United States
(12) Patent Application Publication
(10) Pub. No.: US 2005/0210738 A1
(43) Pub. Date: Sep. 29, 2005

(54) Preparation of levulinic acid esters from alpha-angelica lactone and alcohols

(75) Inventor: Leo Ernest Manzer, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(73) Assignee: E. I. DU PONT DE NEMOURS AND COMPANY

(21) Appl. No.: 11/087,926

(22) Filed: Mar. 23, 2005

Related U.S. Application Data
(60) Provisional application No. 60/556,226, filed on Mar. 24, 2004.

(51) Int. Cl. C07C 69/74; C10L 1/18

(52) U.S. Cl. 44/385; 560/1

(57) ABSTRACT

The present invention relates to a process for producing levulinic acid esters from α-angelica lactone and alcohols in the presence of a basic catalyst. In addition, a method is described herein for producing fuel or fuel additives comprising levulinic acid esters produced from α-angelica lactone and alcohol. In addition, compositions are described comprising levulinic acid esters for use as fuel or fuel additives.

### FIELD OF THE INVENTION

This invention relates to a process for producing levulinic acid esters from α-angelica lactone and alcohols in the presence of a basic catalyst. This invention also relates to the use of levulinic acid esters as fuel and fuel additives for gasoline fuel, diesel fuel and biofuel.

### BACKGROUND OF THE INVENTION

Levulinic acid esters are useful as solvents, plasticizers, flavoring agents and chemical intermediates. Levulinic acid esters also exhibit characteristics that make them particularly suitable for use as oxygenate additives for diesel fuel, gasoline fuel and biofuel, octane number-enhancing agents for gasoline, and as cetane number-enhancing agents in diesel fuels. The commercial use of levulinic acid esters has been limited due to the high cost of production. The production of levulinic acid esters from renewable resources, such as cellulosic biomass, however, represents a potentially low-cost route to the manufacture of these esters. The production of levulinic acid esters from biomass-derived levulinic acid is described, for example, in commonly assigned U.S. Patent Application 60/369,380, filed on Apr. 1, 2003 and Ser. No. 10/768,276, filed on Jan. 30, 2004.

The present invention describes the synthesis of levulinic acid esters from α-angelica lactone and alcohol in the presence of basic catalysts. The synthesis of esters of levulinic acid from α-angelica lactone and alcohol in the presence of homogeneous strong acids, such as hydrogen chloride and sulfuric acid, has been described by Langlois and Wolff (JACS (1948) 70:2624, U.S. Pat. No. 2,493,676) and Iwakura, et al. (Kogyo Kagaku Zasshi (1956) 59:476). The synthesis of levulinic acid esters from α-angelica lactone and alcohol in the presence of basic catalysts has not previously been demonstrated. The reaction of α-angelica lactone with some alcohols is enhanced in the presence of a basic catalyst; indeed, it is shown by the present invention that the conversion of α-angelica lactone and phenol to phenyl levulinate results in much higher yields and reduced tarry residue when the reaction is catalyzed by a basic catalyst rather than an acidic catalyst.

### SUMMARY OF THE INVENTION

Described herein is a process for producing at least one levulinic acid ester from a reaction of α-angelica lactone with alcohol. The process, shown by the equation below, comprises contacting α-angelica lactone with at least one alcohol in the presence of a basic catalyst, said basic 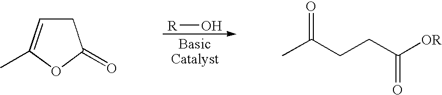
catalyst being optionally supported on a catalyst support:
wherein:
   (i) R is an alkyl, aryl or alkaryl hydrocarbyl group having from one to twenty carbons, and wherein R may be C₁-C₂₀ unsubstituted or substituted alkyl, C₂-C₂₀ unsubstituted or substituted alkenyl, C₂-C₂₀ unsubstituted or substituted alkynyl, C₃-C₂₀ unsubstituted or substituted cycloalkyl, C₃-C₂₀ unsubstituted or substituted cycloalkyl containing at least one heteroatom, C₆-C₂₀ unsubstituted or substituted aryl, C₆-C₂₀ unsubstituted or substituted aryl containing at least one heteroatom, C₇-C₂₀ unsubstituted or substituted alkaryl, or C₇-C₂₀ unsubstituted or substituted alkaryl containing at least one heteroatom; and
   (ii) said at least one levulinic acid ester is optionally recovered.

The basic catalyst may be a metal catalyst selected from the group consisting of metal silicates, metal carbonates, metal bicarbonate, metal oxides, metal hydroxides, metal phosphates, metal aluminates, metal carboxylates and combinations thereof, said metal being selected from the group consisting of Group I and Group II elements of the Periodic Table. The metal of the basic metal catalyst may be selected from the group consisting of cesium, rubidium, lithium, sodium, potassium, strontium, scandium, calcium, barium, magnesium, compounds thereof and combinations thereof. The basic metal catalyst may optionally be supported on a catalyst support. The basic catalyst may also be a homogeneous catalyst selected from the following groups: 1) trialkyl, tricycloalkyl, triaryl and trialkaryl amine, 2) trialkyl, tricycloalkyl, triaryl and trialkaryl diamine, 3) trialkyl, tricycloalkyl, triaryl and trialkaryl arsine, 4) trialkyl, tricycloalkyl, triaryl and trialkaryl diarsine, 5) trialkyl, tricycloalkyl, triaryl and trialkaryl phosphine, 6) trialkyl, tricycloalkyl, triaryl and trialkaryl diphosphine, 7) salts of groups 1-6, and 8) combinations of groups 1-7.

The molar ratio of alcohol to α-angelica lactone may be approximately 1:1 or greater than 1:1. The process of the invention is performed at a temperature of from about 1° C. to about 300° C., and a pressure of from about 0.1 MPag to about 15 MPag. Typically, the amount of catalyst used is from about 0.1% to about 50% by weight of the solution comprising the reactants.

The present invention also provides compositions comprising levulinic acid esters made by the process described above for use as fuels and fuel additives.

### DETAILED DESCRIPTION OF THE INVENTION

The present invention relates to a process for producing levulinic acid esters from α-angelica lactone and alcohols in the presence of a basic catalyst. In addition, a method is described herein for producing fuel additives comprising levulinic acid esters derived from α-angelica lactone and alcohol for use as oxygenates. Besides being used as oxygenate additives for diesel fuel, gasoline fuel and biofuel, the esters of the invention can also be used as octane number-enhancing agents for gasoline, and as cetane number-enhancing agents in diesel fuels. The reaction mixture of esters of the present invention can also be directly used as 100% fuel.

Definitions

In this disclosure, a number of terms and abbreviations are used. The following definitions are provided.

“Hydrocarbyl” refers to monovalent groups of atoms containing only carbon and hydrogen, and may be chiral or achiral. Unless otherwise stated, it is preferred in the method of the invention that hydrocarbyl (and substituted hydrocarbyl) groups contain 1 to 20 carbon atoms.

“Aliphatic” refers to a group of organic chemical compounds in which the carbon atoms are linked in open chains.

“Alkyl” refers to an alkyl group up to and including 20 carbons. Common examples of alkyl groups include methyl, ethyl, propyl, isopropyl, butyl, s-butyl, isobutyl, pentyl, neopentyl, hexyl, heptyl, isoheptyl, 2-ethylhexyl, cyclohexyl and octyl.

As used herein, the term “aryl” denotes aromatic cyclic groups including, but not limited to, phenyl groups. An “aromatic group” is benzene or compounds that resemble benzene in chemical behavior. Common examples of aryl groups include benzene, biphenyl, terphenyl, naphthalene, phenyl naphthalene, and naphthylbenzene.

A “heteroatom” is an atom other than carbon in the structure of a heterocyclic compound. A heterocyclic compound is a compound containing more than one kind of atom joined in a ring.

“Substituted” refers to a group attached to a reactant containing one or more substituent groups that do not cause the compound to be unstable or unsuitable for the use of reaction intended. Substituent groups useful in the method of the invention include nitrile, ether, ester, halo, amino (including primary, secondary and tertiary amino), hydroxy, oxo, vinylidene or substituted vinylidene, silyl or substituted silyl, nitro, nitroso, sulfinyl, sulfonyl, alkali metal salt, boranyl or substituted boranyl, and thioether groups.

“Selectivity” refers to the weight percent of a particular reaction product in the total product weight (including the weight of unreacted reactants).

“Conversion” refers to the weight percent of a particular reactant that is converted to product.

“α-Angelica lactone” as used herein means a compound having the following formula:

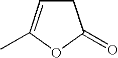

An “alcohol” of the invention is a compound having the Formula “R—OH” wherein R is an alkyl, aryl or alkaryl hydrocarbyl group having from one to twenty carbons, and wherein R may be C₁-C₂₀ unsubstituted or substituted alkyl, C₂-C₂₀ unsubstituted or substituted alkenyl, C₂-C₂₀ unsubstituted or substituted alkynyl, C₃-C₂₀ unsubstituted or substituted cycloalkyl, C₃-C₂₀ unsubstituted or substituted cycloalkyl containing at least one heteroatom, C₆-C₂₀ unsubstituted or substituted aryl, C₆-C₂₀ unsubstituted or substituted aryl containing at least one heteroatom, C₇-C₂₀ unsubstituted or substituted alkaryl, or C₇-C₂₀ unsubstituted or substituted alkaryl containing at least one heteroatom.

A “levulinic acid ester” of the invention is an ester having the formula exemplified below, wherein R is an alkyl, aryl or alkaryl hydrocarbyl group having from one to twenty carbons, and wherein R may be C₁-C₂₀ unsubstituted or substituted alkyl, C₂-C₂₀ unsubstituted or substituted alkenyl, C₂-C₂₀ unsubstituted or substituted alkynyl, C₃-C₂₀ unsubstituted or substituted cycloalkyl, C₃-C₂₀ unsubstituted or substituted cycloalkyl containing at least one heteroatom, C₆-C₂₀ unsubstituted or substituted aryl, C₆-C₂₀ unsubstituted or substituted aryl containing at least one heteroatom, C₇-C₂₀ unsubstituted or substituted alkaryl, or C₇-C₂₀ unsubstituted or substituted alkaryl containing at least one heteroatom:

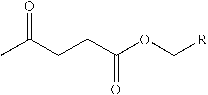

A “metal catalyst” is a catalyst that comprises one or more metals. A “basic metal catalyst” is a catalyst that comprises one or more metals and that acts as a basic catalyst as described herein. A “supported metal catalyst” is a catalyst comprising at least one metal and at least one support.

By the term “biofuel” is meant either a 100% biodiesel or a mixture comprising biodiesel and regular petroleum-based diesel from a refinery. For example, B20 is a mixture of 20% biodiesel based on vegetable oil, and 80% regular diesel. Biodiesel is a product of esterification of oils such as palm, canola, tallow, corn, and soy, with methanol.

By the term “octane number” is meant an empirical rating of the anti-knock quality of a fuel. “Knock” is caused by secondary ignition of fuel unburned after normal spark ignition, which gives rise to a fast moving flame front in an automobile's engine cylinder. Pressure waves are setup, which vibrate against the cylinder walls giving rise to a “knocking” sound. This feature of fuel is undesirable because it accelerates wear in the engine bearings and causes overheating in the cylinders. The tendency of the fuel to knock increases as the compression ratio increases. Certain fuels have better anti-knock characteristics than others because of their molecular structure, branched structures having better characteristics. On the arbitrary octane scale, iso-octane (C₈H₁₈) is given an octane value of 100; n-heptane (C₇H₁₆) is given a value of zero. The octane number of a fuel is determined by comparing its performance in a standard spark-ignition engine with the performance of various mixtures of iso-octane and n-heptane. The behavior of the fuel is carefully matched by a known mixture of iso-octane and n-heptane. The percentage of isooctane in this mixture is then taken as the octane number of the fuel.

The “cetane number” is used to evaluate fuels used in compression-ignition (diesel) engines and is analogous to octane number. Cetane (n-hexadecane, C₁₆H₃₄) is designated 100 and alpha-methyl-naphthalene (C₁₁H₁₀) as zero, so that the cetane number of a fuel is the proportion of the cetane in the mixture of these having the same ignition delay after injection of the fuel as the test fuel.

The invention described herein provides a process for preparing levulinic acid (or levulinate) esters from α-angelica lactone. The process comprises contacting α-angelica lactone with at least one alcohol in the presence of a basic catalyst:

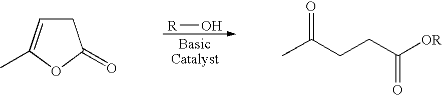

α-Angelica lactone for use in the process of the invention may be obtained by vacuum distilling levulinic acid as described in U.S. Pat. No. 2,809,203, or may be derived from biomass as described in U.S. patent application Ser. No. ______ “CL-2406”.

In the present invention, R is an alkyl, aryl or alkaryl hydrocarbyl group having from one to twenty carbons, and R may be C₁-C₂₀ unsubstituted or substituted alkyl, C₂-C₂₀ unsubstituted or substituted alkenyl, C₂-C₂₀ unsubstituted or substituted alkynyl, C₃-C₂₀ unsubstituted or substituted cycloalkyl, C₃-C₂₀ unsubstituted or substituted cycloalkyl containing at least one heteroatom, C₆-C₂₀ unsubstituted or substituted aryl, C₆-C₂₀ unsubstituted or substituted aryl containing at least one heteroatom, C₇-C₂₀ unsubstituted or substituted alkaryl, or C₇-C₂₀ unsubstituted or substituted alkaryl containing at least one heteroatom.

A basic catalyst, with or without a support, is present in the process of the invention to effect the conversion of α-angelica lactone to at least one levulinic acid ester. A promoter may optionally be used to aid the reactions; the promoter may be a metal.

Typical alcohols of the invention are aliphatic alcohols having from one to ten carbons or aryl or alkaryl alcohols having from six to 13 carbons. Typical alcohols include, but are not limited to, methanol, ethanol, propanol, i-propanol, n-butanol, i-butanol, t-butanol, cyclohexanol, phenol and benzyl alcohol. Mixtures of alcohols may also be used, resulting in a mixture of levulinic acid esters as the product.

In one embodiment of the invention, the molar ratio of alcohol to α-angelica lactone is greater than 1:1 (alcohol:α-angelica lactone); in another embodiment of the invention, the molar ratio of alcohol to α-angelica lactone is about 1:1.

In one embodiment, the temperature range for the process of the invention is from about 1° C. to about 300° C. In another embodiment, the temperature range for the reaction is from about 25° C. to about 200° C.

In one embodiment, the pressure range for the reaction is from about 0.1 MPag to about 15 MPag. In another embodiment, the pressure range is from about 0.1 MPag to about 10 MPag.

The catalyst useful in the invention is a substance that affects the rate of the reaction but not the reaction equilibrium, and emerges from the process chemically unchanged. A suitable basic catalyst of the invention can be defined either as a substance which has the ability to accept protons as defined by Brönsted, or as a substance which has an unshared electron pair with which it can form a covalent bond with an atom, molecule or ion as defined by Lewis. A further definition of basic catalysts and how to determine if a particular substance is basic can be found in Tanabe, K., Catalysis: Science and Technology (Anderson, J. and Boudart, M. (eds.) (1981) Vol. 2, pages 232-273, Springer-Verlag, N.Y.).

One group of suitable basic catalysts includes metal catalysts, which include, but are not limited to, metal silicates, metal carbonates, metal bicarbonates, metal oxides, metal hydroxides, metal phosphates, metal aluminates, metal carboxylates and combinations thereof. The metals of the basic metal catalysts may be selected from the group consisting of Group I and Group II elements of the Periodic Table. In one embodiment of the invention, the metals may be selected from the group consisting of cesium, rubidium, lithium, sodium, potassium, strontium, scandium, calcium, barium and magnesium, salts thereof and mixtures thereof. The metal catalysts of the invention may be obtained from commercial manufacturers, or they can be prepared from suitable starting materials using methods known in the art. Typically, the amount of catalyst used is from about 0.1% to about 50% by weight of the solution comprising the reactants.

The basic metal catalysts employed herein may be used as powders, granules, or other particulate forms, or may be supported on an essentially inert support as is common in the art of catalysis. Selection of an optimal average particle size for the catalyst will depend upon such process parameters as reactor residence time and desired reactor flow rates.

The basic metal catalyst useful in the invention may be supported or unsupported. A supported catalyst is one in which the active catalyst agent is deposited on a support material by a number of methods, such as spraying, soaking or physical mixing, followed by drying, calcination, and if necessary, activation through methods such as reduction or oxidation. Materials frequently used as a support are porous solids with high total surface areas (external and internal), which can provide high concentrations of active sites per unit weight of catalyst. The catalyst support may enhance the function of the catalyst agent. A supported metal catalyst is a supported catalyst in which the catalyst agent is a metal.

Suitable supports include but are not limited to carbon, alumina, silica, silica-alumina, silica-titania, silica-zirconia, titania, titania-alumina, zirconia, barium sulfate, calcium carbonate, strontium carbonate, zeolites, compounds thereof and combinations thereof. The support can be neutral, acidic or basic, as long as the surface of the catalyst/support combination is basic. In one embodiment of the invention, the support is selected from the group consisting of silica, titania, zirconia, silica-titania and silica-zirconia. Preferred supports are those, which are neutral and have low surface areas. Commonly used techniques for treatment of supports with metal catalysts can be found in B. C. Gates, Heterogeneous Catalysis, Vol. 2, pp. 1-29, Ed. B. L. Shapiro, Texas A & M University Press, College Station, Tex., 1984.

Combinations of catalyst and support system may include any one of the metals referred to herein with any of the supports referred to herein. Preferred combinations of catalyst and support include cesium on silica, cesium on titania, cesium on zirconia, cesium on silica-titania, cesium on silica-zirconia, rubidium on silica, rubidium on titania, rubidium on zirconia, rubidium on silica-titania, rubidium on silica-zirconia, lithium on silica, lithium on titania, lithium on zirconia, lithium on silica-titania, lithium on silica-zirconia, sodium on silica, sodium on titania, sodium on zirconia, sodium on silica-titania, sodium on silica-zirconia, potassium on silica, potassium on titania, potassium on zirconia, potassium on silica-titania, potassium on silica-zirconia, strontium on silica, strontium on titania, strontium on zirconia, strontium on silica-titania, strontium on silica-zirconia, scandium on silica, scandium on titania, scandium on zirconia, scandium on silica-titania, scandium on silica-zirconia, calcium on silica, calcium on titania, calcium on zirconia, calcium on silica-titania, calcium on silica-zirconia, barium on silica, barium on titania, barium on zirconia, barium on silica-titania, barium on silica-zirconia, magnesium on silica, magnesium on titania, magnesium on zirconia, magnesium on silica-titania and magnesium on silica-zirconia.

In the process of the invention, the preferred content of the metal catalyst in the supported catalyst is from about 0.1% to about 40% of the supported catalyst based on metal catalyst weight plus the support weight. A more preferred metal catalyst content range is from about 10% to about 20% of the supported catalyst. A further preferred metal catalyst content range is from about 7% to about 15% of the supported catalyst. Typically the supported catalyst is used at from about 1% to about 50% by weight of the solution. In another embodiment, the supported catalyst is used at from about 1% to about 10%.

The basic metal catalysts of the present invention may further comprise catalyst promoters, which may enhance the efficiency of the metal catalyst. Use of these materials is common and well known in the art (see, for example, Kirk-Othmer Encyclopedia of Chemical Technology (Howe-Grant (ed.) (1993) Vol. 5, pages 326-346, John Wiley & Sons, New York) and Ullmann's Encyclopedia of Industrial Chemistry (Gerhartz, et al., (eds.) (1986) Vol. A5, pages 337-346, VCH Publishers, New York). Catalyst promoters may be selected from metals from Group I and Group II of the Periodic Table. The relative percentages of the catalyst promoter may vary. Useful amounts of promoter can be from about 0.01% to about 50% by weight of catalyst. The promoter herein may be incorporated into the catalyst during any step in the chemical processing of the catalyst constituent. The chemical promoter generally enhances the physical or chemical function of the catalyst agent, but can also be added to retard undesirable side reactions.

An additional group of suitable catalysts for the process of the invention includes homogeneous catalysts. Homogeneous catalysts useful for the invention are selected from the following groups: 1) trialkyl, tricycloalkyl, triaryl and trialkaryl amine, 2) trialkyl, tricycloalkyl, triaryl and trialkaryl diamine, 3) trialkyl, tricycloalkyl, triaryl and trialkaryl arsine, 4) trialkyl, tricycloalkyl, triaryl and trialkaryl diarsine, 5) trialkyl, tricycloalkyl, triaryl and trialkaryl phosphine, 6) trialkyl, tricycloalkyl, triaryl and trialkaryl diphosphine, 7) salts of groups 1-6, and 8) combinations of groups 1-7. Examples of suitable homogeneous catalysts include, but are not limited to, trimethylamine, triethylamine and Dabco™, tricyclohexylphosphine, dimethylbenzylphosphine, diethylphenylphosphine, ethyldiphenylarsine, tricyclohexylarsine, bis(dimethylphosphino)ethane. Typically, homogeneous catalysts are used at from about 1% to about 50% by weight of the solution.

The process of the present invention may be carried out in batch, sequential batch (i.e., a series of batch reactors) or in continuous mode in any of the equipment customarily employed for continuous processes (see for example, H. S. Fogler, Elementary Chemical Reaction Engineering, Prentice-Hall, Inc., N.J., USA).

It will be appreciated that the selectivities and yields of product may be enhanced by additional contact with the catalyst. For example, yields and selectivities may be increased where the reactor effluent containing a mixture of reactant and product may be contacted additional times over the catalyst under the reaction conditions set forth herein to enhance the conversion of reactant to product.

The process of the instant invention may additionally comprise the recovery or isolation of one or more of the levulinic acid esters. This can be done by any method known in the art, such as distillation, decantation, recrystallization, extraction or chromatography.

Compositions comprising levulinic acid esters produced by the process of the invention are useful as fuel additives. The levulinic acid esters may optionally be recovered as reaction products of the process of the invention for use as fuel additives, or the non-purified product mixture produced in the process of the invention, which may comprise unreacted alcohol, may be used directly. Besides being used as oxygenate additives for diesel fuel, gasoline fuel and biofuel, these esters can also be used as octane number-enhancing agents for gasoline, and as cetane number-enhancing agents in diesel fuels. The reaction mixture of esters of the present invention can also be directly used as 100% fuel.

“Fuel additives” are substances that can improve the fuel efficiency of an engine, for example, as measured by the octane number, the cetane number or any other index suited to measure the efficiency of a particular fuel. A fuel additive may also perform the function of lubricating, cleaning and stabilizing the fuel and may improve performance, economy, and injector life, reduce emissions, reduce smoke related to an engine, help eliminate tank draining, lower a gel point of the fuel or provide a clean burning fuel that can inhibit polluting agents in emissions.

“Oxygenates” is a commonly referred to group of chemical compounds that raise the oxygen content of gasoline. Oxygen helps gasoline burn more completely, reducing harmful tailpipe emissions from motor vehicles. In one respect, the oxygen dilutes or displaces gasoline components such as aromatics (e.g., benzene) and sulfur. Additionally, it optimizes oxidation during combustion. Most gasoline suppliers meet the oxygen content requirements of the different clean fuel programs by adding oxygenate fuel additives, most commonly methyl tertiary-butyl ether (hereinafter referred to as MTBE), to gasoline blend stocks. Recently, various environmental protection agencies have begun raising concerns regarding the detection of MTBE in surface and ground water.

Levulinic acid esters of the invention with low water solubility can be used to meet governmental oxygen requirements for gasoline and oxy-gasoline fuels. These low solubility esters would have a reduced solubility in surface and subsurface water and could therefore reduce the impact on such waters from spills and emissions of oxygenated fuels. As shown in U.S. Patent Application 60/369,380, esters of the present invention, such as ethyl levulinate and methyl levulinate, have a significantly higher oxygen content than MTBE. Therefore, a lesser amount of esters is required to meet the various clean fuel programs' oxygen requirements for gasoline. Thus, the present invention provides compositions of levulinic acid esters or mixtures of levulinic acid esters produced by the process of the invention for use as fuel additives, such as oxygenates for gasoline, octane number-enhancing agents for gasoline, oxygenates for diesel, cetane number-enhancing agents for diesel or fuel additives for biofuel. The present invention also provides compositions of levulinic acid esters or mixtures of levulinic acid esters produced by the process of the invention for use as fuel.

As liquid organic based fuels for use in internal combustion engines, the reaction mixture containing levulinic acid esters or mixtures of levulinic acid esters as obtained from the process of the invention, can be used in the range of from about 1% to about 99% by volume, as additive to gasoline, diesel, or biofuel. A preferred range is from about 1% to about 90% by volume. A more preferred range is from about 1% to about 50% by volume. A further preferred range is from about 1% to about 20% by volume. Moreover, the reaction mixtures of esters of this invention can also be used as 100% fuel.

The invention is further demonstrated by the following Examples.

### EXAMPLES

In the following examples, “OAc” refers to acetate; “Oct3P” refers to trioctyl phosphine; GC is gas chromatograph; the unit of pressure Mpag refers to MPa gage, and “temp” refers to temperature.

For catalyst preparation a commercially available support such as carbon, alumina, silica, silica-alumina or titania was impregnated by incipient wetness with a metal salt (20% by weight of the metal salt). The metals used were the acetate salts of cesium, rubidium, lithium, potassium, magnesium, calcium, strontium and barium.

In the following examples, triethylamine, Li₂CO₃, Na₂CO₃, K₂CO₃, ScCO₃, CsCO₃, cesium acetate, rubidium acetate, lithium acetate, potassium acetate, magnesium acetate, calcium acetate, strontium acetate, barium acetate, H₂SO₄, Amberlyst® 15 and Zn(BF₄)₂ were obtained from Alfa Aesar (Ward Hill, Mass.); Dabco™ was obtained from Sigma-Aldrich (St. Louis, Mo.). SiO₂ (Grade 55) and SiO₂ (1700) was obtained from W. R. Grace (Columbia, Md.); KA-160-SiO₂ was obtained from Engelhard Corp. (Iselin, N.J.).

### Example 1

### General Procedure for the Reaction of Alcohols and α-Angelica Lactone

A 2 cc pressure vessel was charged with 700 mg of a solution consisting of alcohol, α-angelica lactone and 50 mg of a catalyst. The reactor was pressurized with nitrogen and heated to reactor temperature for a specified period of time. The vessel was then cooled, vented and the products analyzed by gas chromatography on a HP-6890 GC (Agilent Technologies; Palo Alto, Calif.) and HP-5972A GC-MS detector equipped with a 25M×0.25MM ID CP-Wax 58 (FFAP) column. The GC yields were obtained by adding methoxyethyl ether as the internal standard.

The examples described below were performed according to a similar procedure under the conditions indicated for each example.

### Examples 2-19

Reaction of α-Angelica Lactone (AGL) with 1-Butanol (1-BuOH) to
Produce Butyl Levulinate (BuLV)

| | | | | N₂ | | AGL | BuLV |
|---|---|---|---|---|---|---|---|
| Expt. | | Time | Temp | Pressure | | Conversion | Selectivity |
| No. | Basic Catalyst | (hrs) | (° C.) | (MPag) | Feedstock | (%) | (%) |
| 2 | Et₃N | 3 | 100 | 5.52 | AGL (30%)/1- | 77.56 | 29.08 |
| | | | | | BuOH (70%) | | |
| 3 | Et₃N | 1 | 150 | 5.52 | AGL (30%)/1- | 89.03 | 45.68 |
| | | | | | BuOH (70%) | | |
| 4 | Et₃N | 3 | 150 | 5.52 | AGL (30%)/1- | 98.88 | 51.31 |
| | | | | | BuOH (70%) | | |
| 5 | Dabco ™ | 3 | 150 | 5.52 | AGL (30%)/1- | 99.22 | 58.01 |
| | | | | | BuOH (70%) | | |
| 6 | 20% | 3 | 150 | 5.52 | AGL (30%)/1- | 96.97 | 55.68 |
| | CsOAc/KA-160 | | | | BuOH (70%) | | |
| | SiO₂ | | | | | | |
| 7 | 20% | 3 | 150 | 5.52 | AGL (30%)/1- | 98.96 | 53.36 |
| | RbOAc/KA-160 | | | | BuOH (70%) | | |
| | SiO₂ | | | | | | |
| 8 | 20% | 1 | 150 | 5.52 | AGL (30%)/1- | 91.87 | 37.56 |
| | CsOAc/KA-160 | | | | BuOH (70%) | | |
| | SiO₂ | | | | | | |
| 9 | 20% | 1 | 150 | 5.52 | AGL (30%)/1- | 95.42 | 42.01 |
| | RbOAc/KA-160 | | | | BuOH (70%) | | |
| | SiO₂ | | | | | | |
| 10 | 20% | 3 | 100 | 5.52 | AGL (30%)/1- | 62.59 | 20.94 |
| | CsOAc/KA-160 | | | | BuOH (70%) | | |
| | SiO₂ | | | | | | |
| 11 | 20% | 3 | 100 | 5.52 | AGL (30%)/1- | 63.21 | 21.98 |
| | RbOAc/KA-160 | | | | BuOH (70%) | | |
| | SiO₂ | | | | | | |
| 12 | Li₂CO₃ | 3 | 100 | 5.52 | AGL (30%)/1- | 22.52 | 79.06 |
| | | | | | BuOH (70%) | | |
| 13 | Na₂CO₃ | 3 | 100 | 5.52 | AGL (30%)/1- | 71.73 | 32.81 |
| | | | | | BuOH (70%) | | |
| 14 | K₂CO₃ | 3 | 100 | 5.52 | AGL (30%)/1- | 59.17 | 62.23 |
| | | | | | BuOH (70%) | | |
| 15 | Sc₂CO₃ | 3 | 100 | 5.52 | AGL (30%)/1- | 33.81 | 64.46 |
| | | | | | BuOH (70%) | | |
| 16 | Li₂CO₃ | 1 | 150 | 5.52 | AGL (30%)/1- | 62.91 | 82.62 |
| | | | | | BuOH (70%) | | |
| 17 | Na₂CO₃ | 1 | 150 | 5.52 | AGL (30%)/1- | 97.30 | 68.86 |
| | | | | | BuOH (70%) | | |
| 18 | K₂CO₃ | 1 | 150 | 5.52 | AGL (30%)/1- | 97.65 | 65.32 |
| | | | | | BuOH (70%) | | |
| 19 | Sc₂CO₃ | 1 | 150 | 5.52 | AGL (30%)/1- | 79.92 | 70.51 |
| | | | | | BuOH (70%) | | |

### Examples 20-23

Reaction of α-Angelica Lactone (AGL) with Cyclohexanol
(CyHxOH) to Produce Cyclohexyl Levulinate (CyHxLV)

| | | | | N₂ | | AGL | CyHxLV |
|---|---|---|---|---|---|---|---|
| Expt. | Basic | Time | Temp | Pressure | | Conversion | Selectivity |
| No. | Catalyst | (hrs) | (° C.) | (MPag) | Feedstock | (%) | (%) |
| 20 | Li₂CO₃ | 1 | 150 | 5.52 | AGL | 37.56 | 38.03 |
| | | | | | (30%)/CyHxOH | | |
| | | | | | (70%) | | |
| 21 | Na₂CO₃ | 1 | 150 | 5.52 | AGL | 79.81 | 14.82 |
| | | | | | (30%)/CyHxOH | | |
| | | | | | (70%) | | |
| 22 | K₂CO₃ | 1 | 150 | 5.52 | AGL | 94.07 | 2.32 |
| | | | | | (30%)/CyHxOH | | |
| | | | | | (70%) | | |
| 23 | Cs₂CO₃ | 1 | 150 | 5.52 | AGL | 80.60 | 10.28 |
| | | | | | (30%)/CyHxOH | | |
| | | | | | (70%) | | |

### Examples 24-41

Reaction of α-Angelica Lactone (AGL) with Methanol
(MeOH) to Produce Methyl Levulinate (MeLV)

| | | | | N₂ | | AGL | MeLV |
|---|---|---|---|---|---|---|---|
| Expt. | Basic | Time | Temp | Pressure | | Conversion | Selectivity |
| No. | Catalyst | (hrs) | (° C.) | (MPag) | Feedstock | (%) | (%) |
| 24 | Li₂CO₃ | 3 | 100 | 5.52 | AGL (30%)/MeOH | 54.94 | 86.17 |
| | | | | | (70%) | | |
| 25 | Na₂CO₃ | 3 | 100 | 5.52 | AGL (30%)/MeOH | 97.47 | 82.45 |
| | | | | | (70%) | | |
| 26 | K₂CO₃ | 3 | 100 | 5.52 | AGL (30%)/MeOH | 90.87 | 81.13 |
| | | | | | (70%) | | |
| 27 | Cs₂CO₃ | 3 | 100 | 5.52 | AGL (30%)/MeOH | 63.68 | 80.47 |
| | | | | | (70%) | | |
| 28 | 20% | 1 | 150 | 5.52 | AGL (30%)/MeOH | 100.00 | 24.53 |
| | LiOAc/SiO₂ | | | | (70%) | | |
| | (1700) | | | | | | |
| 29 | 20% | 1 | 150 | 5.52 | AGL (30%)/MeOH | 100.00 | 67.92 |
| | KOAc/SiO₂ | | | | (70%) | | |
| | (1700) | | | | | | |
| 30 | 20% | 1 | 150 | 5.52 | AGL (30%)/MeOH | 96.49 | 69.05 |
| | CsOAc/SiO₂ | | | | (70%) | | |
| | (1700) | | | | | | |
| 31 | 20% | 1 | 150 | 5.52 | AGL (30%)/MeOH | 100.00 | 4.32 |
| | MgOAc/SiO₂ | | | | (70%) | | |
| | (1700) | | | | | | |
| 32 | 20% | 1 | 150 | 5.52 | AGL (30%)/MeOH | 100.00 | 70.14 |
| | CaOAc/SiO₂ | | | | (70%) | | |
| | (1700) | | | | | | |
| 33 | 20% | 1 | 150 | 5.52 | AGL (30%)/MeOH | 93.77 | 69.22 |
| | SrOAc/SiO₂ | | | | (70%) | | |
| | (1700) | | | | | | |
| 34 | 20% | 1 | 150 | 5.52 | AGL (30%)/MeOH | 95.82 | 56.66 |
| | BaOAc/SiO₂ | | | | (70%) | | |
| | (1700) | | | | | | |
| 35 | 20% | 5 | 100 | 5.52 | AGL (30%)/MeOH | 97.70 | 79.57 |
| | LiOac/SiO₂ | | | | (70%) | | |
| | (1700) | | | | | | |
| 36 | 20% | 5 | 100 | 5.52 | AGL (30%)/MeOH | 83.10 | 74.91 |
| | KOAc/SiO₂ | | | | (70%) | | |
| | (1700) | | | | | | |
| 37 | 20% | 5 | 100 | 5.52 | AGL (30%)/MeOH | 71.25 | 71.37 |
| | CsOAc/SiO₂ | | | | (70%) | | |
| | (1700) | | | | | | |
| 38 | 20% | 5 | 100 | 5.52 | AGL (30%)/MeOH | 95.38 | 74.47 |
| | MgOAc/SiO₂ | | | | (70%) | | |
| | (1700) | | | | | | |
| 39 | 20% | 5 | 100 | 5.52 | AGL (30%)/MeOH | 97.85 | 33.48 |
| | CaOAc/SiO₂ | | | | (70%) | | |
| | (1700) | | | | | | |
| 40 | 20% | 5 | 100 | 5.52 | AGL (30%)/MeOH | 84.04 | 70.26 |
| | SrOAc/SiO₂ | | | | (70%) | | |
| | (1700) | | | | | | |
| 41 | 20% | 5 | 100 | 5.52 | AGL (30%)/MeOH | 82.24 | 71.95 |
| | BaOAc/SiO₂ | | | | (70%) | | |
| | (1700) | | | | | | |

### Examples 42-52

Reaction of α-Angelica Lactone (AGL) with
Ethanol (EtOH) to Produce Ethyl Levulinate (EtLV)

| | | | | N₂ | | AGL | EtLV |
|---|---|---|---|---|---|---|---|
| Expt. | Basic | Time | Temp | Pressure | | Conversion | Selectivity |
| No. | Catalyst | (hrs) | (° C.) | (MPag) | Feedstock | (%) | (%) |
| 42 | Li₂CO₃ | 3 | 100 | 5.52 | AGL | 44.64 | 47.33 |
| | | | | | (30%)/EtOH | | |
| | | | | | (70%) | | |
| 43 | Na₂CO₃ | 3 | 100 | 5.52 | AGL | 58.30 | 56.30 |
| | | | | | (30%)/EtOH | | |
| | | | | | (70%) | | |
| 44 | K₂CO₃ | 3 | 100 | 5.52 | AGL | 57.50 | 57.04 |
| | | | | | (30%)/EtOH | | |
| | | | | | (70%) | | |
| 45 | Cs₂CO₃ | 3 | 100 | 5.52 | AGL | 42.97 | 45.78 |
| | | | | | (30%)/EtOH | | |
| | | | | | (70%) | | |
| 46 | 20% | 1 | 150 | 5.52 | AGL | 97.31 | 71.91 |
| | LiOAc/SiO₂ | | | | (30%)/EtOH | | |
| | (1700) | | | | (70%) | | |
| 47 | 20% | 1 | 150 | 5.52 | AGL | 96.48 | 62.03 |
| | KOAc/SiO₂ | | | | (30%)/EtOH | | |
| | (1700) | | | | (70%) | | |
| 48 | 20% | 1 | 150 | 5.52 | AGL | 89.19 | 66.65 |
| | CsOAc/SiO₂ | | | | (30%)/EtOH | | |
| | (1700) | | | | (70%) | | |
| 49 | 20% | 1 | 150 | 5.52 | AGL | 95.88 | 70.18 |
| | MgOAc/SiO₂ | | | | (30%)/EtOH | | |
| | (1700) | | | | (70%) | | |
| 50 | 20% | 1 | 150 | 5.52 | AGL | 95.30 | 51.94 |
| | CaOAc/SiO₂ | | | | (30%)/EtOH | | |
| | (1700) | | | | (70%) | | |
| 51 | 20% | 1 | 150 | 5.52 | AGL | 68.01 | 54.12 |
| | SrOAc/SiO₂ | | | | (30%)/EtOH | | |
| | (1700) | | | | (70%) | | |
| 52 | 20% | 1 | 150 | 5.52 | AGL | 69.15 | 52.61 |
| | BaOAc/SiO₂ | | | | (30%)/EtOH | | |
| | (1700) | | | | (70%) | | |

### Examples 53-59

Reaction of α-Angelica Lactone (AGL) with Phenol
(PhOH) to Produce Phenyl Levulinate (PhLV)

| | | | | N2 | | AGL | PhLV |
|---|---|---|---|---|---|---|---|
| Expt. | Basic | Time | Temp | Pressure | | Conversion | Selectivity |
| No. | Catalyst | (hrs) | (° C.) | (MPag) | Feedstock | (%) | (%) |
| 53 | 20% | 1 | 150 | 5.52 | AGL (30%)/PhOH | 97.57 | 22.84 |
| | LiOAc/SiO₂ | | | | (70%) | | |
| | (1700) | | | | | | |
| 54 | 20% | 1 | 150 | 5.52 | AGL (30%)/PhOH | 97.89 | 63.04 |
| | KOAc/SiO₂ | | | | (70%) | | |
| | (1700) | | | | | | |
| 55 | 20% | 1 | 150 | 5.52 | AGL (30%)/PhOH | 96.05 | 65.39 |
| | CsOAc/SiO₂ | | | | (70%) | | |
| | (1700) | | | | | | |
| 56 | 20% | 1 | 150 | 5.52 | AGL (30%)/PhOH | 83.86 | 39.80 |
| | MgOAc/SiO₂ | | | | (70%) | | |
| | (1700) | | | | | | |
| 57 | 20% | 1 | 150 | 5.52 | AGL (30%)/PhOH | 92.12 | 75.68 |
| | CaOAc/SiO₂ | | | | (70%) | | |
| | (1700) | | | | | | |
| 58 | 20% | 1 | 150 | 5.52 | AGL (30%)/PhOH | 91.47 | 40.47 |
| | SrOAc/SiO₂ | | | | (70%) | | |
| | (1700) | | | | | | |
| 59 | 20% | 1 | 150 | 5.52 | AGL (30%)/PhOH | 92.16 | 70.83 |
| | BaOAc/SiO₂ | | | | (70%) | | |
| | (1700) | | | | | | |

### Examples 60-62

### Reaction of α-Angelica Lactone (AGL) with Phenol (PhOH) in the Presence of an Basic Catalyst to Produce Phenyl Levulinate (PhLV)

In contrast to the results obtained with basic catalysts (see Experiments 53-59), the reaction of α-angelica lactone with phenol in the presence of an acidic catalyst resulted in a black, tarry residue and poor yield of phenyl levulinate.

| | | | | N2 | | AGL | PhLV |
|---|---|---|---|---|---|---|---|
| Expt. | Acid | Time | Temp | Pressure | | Conversion | Selectivity |
| No. | Catalyst | (hrs) | (° C.) | (MPag) | Feedstock | (%) | (%) |
| 60 | H₂SO₄ | 1 | 150 | 5.52 | AGL | 100.00 | <5% |
| | | | | | (30%)/PhOH | | |
| | | | | | (70%) | | |
| 61 | Amberlyst | 1 | 150 | 5.52 | AGL | 100.00 | <5% |
| | 15 | | | | (30%)/PhOH | | |
| | | | | | (70%) | | |
| 62 | Zn(BF₄)₂ | 1 | 150 | 5.52 | AGL | 100.00 | <5% |
| | | | | | (30%)/PhOH | | |
| | | | | | (70%) | | |

### Examples 63-70

Reaction of α-Angelica Lactone (AGL) with Benzyl
Alcohol (BzOH) to Produce Benzyl Levulinate (BzLV)

| | | | | N₂ | | AGL | BzLV |
|---|---|---|---|---|---|---|---|
| Expt. | Basic | Time | Temp | Pressure | | conversion | Selectivity |
| No. | Catalyst | (hrs) | (° C.) | (MPag) | Feedstock | (%) | (%) |
| 63 | Li₂CO₃ | 1 | 150 | 5.52 | AGL (30%)/BzOH | 87.25 | 46.37 |
| | | | | | (70%) | | |
| 64 | Na₂CO₃ | 1 | 150 | 5.52 | AGL (30%)/BzOH | 96.66 | 45.20 |
| | | | | | (70%) | | |
| 65 | K₂CO₃ | 1 | 150 | 5.52 | AGL (30%)/BzOH | 95.33 | 57.01 |
| | | | | | (70%) | | |
| 66 | Cs₂CO₃ | 1 | 150 | 5.52 | AGL (30%)/BzOH | 87.00 | 51.90 |
| | | | | | (70%) | | |
| 67 | 20% | 1 | 150 | 5.52 | AGL (30%)/BzOH | 95.19 | 49.01 |
| | LiOAc/SiO₂ | | | | (70%) | | |
| | (Grade 55) | | | | | | |
| 68 | 20% | 1 | 150 | 5.52 | AGL (30%)/BzOH | 96.60 | 83.00 |
| | KOAc/SiO₂ | | | | (70%) | | |
| | (1700) | | | | | | |
| 69 | 20% | 1 | 150 | 5.52 | AGL (30%)/BzOH | 91.38 | 54.35 |
| | CsOAc/SiO₂ | | | | (70%) | | |
| | (1700) | | | | | | |
| 70 | 20% | 1 | 150 | 5.52 | AGL (30%)/BzOH | 93.60 | 54.92 |
| | MgOAc/SiO₂ | | | | (70%) | | |
| | (1700) | | | | | | |

### Example 71

Reaction of α-Angelica Lactone (AGL) with Cyclohexanol (CyHxOH) in the
Presence of a Basic Catalyst to Produce Cyclohexyl Levulinate (CyLV)

| | | | | N₂ | | AGL | CyLV |
|---|---|---|---|---|---|---|---|
| Expt. | Basic | Time | Temp | Pressure | | conversion | Selectivity |
| No. | Catalyst | (hrs) | (° C.) | (MPag) | Feedstock | (%) | (%) |
| 71 | Oct3P | 1 | 150 | 5.52 | AGL | 96.41 | 1.68 |
| | | | | | (30%)/CyHxOH | | |
| | | | | | (70%) | | |

### Example 72

Reaction of α-Angelica Lactone (AGL) with Benzyl Alcohol (BzOH) in the
Presence of a Basic Catalyst to Produce Benzyl Levulinate (BzLV)

| | | | | N₂ | | AGL | BzLV |
|---|---|---|---|---|---|---|---|
| Expt. | Basic | Time | Temp | Pressure | | conversion | Selectivity |
| No. | Catalyst | (hrs) | (° C.) | (MPag) | Feedstock | (%) | (%) |
| 72 | Oct3P | 1 | 150 | 5.52 | AGL (30%)/BzOH | 89.3 | 1.72.0 |
| | | | | | (70%) | | |

1. A process for preparing a reaction product comprising at least one levulinic acid ester, the process comprising contacting α-angelica lactone with at least one alcohol in the presence of a basic catalyst, said basic catalyst being optionally supported on a catalyst 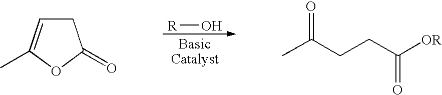
support:
wherein
(iii) R is an alkyl, aryl or alkaryl hydrocarbyl group having from one to twenty carbons, and wherein R may be C₁-C₂₀ unsubstituted or substituted alkyl, C₂-C₂₀ unsubstituted or substituted alkenyl, C₂-C₂₀ unsubstituted or substituted alkynyl, C₃-C₂₀ unsubstituted or substituted cycloalkyl, C₃-C₂₀ unsubstituted or substituted cycloalkyl containing at least one heteroatom, C₆-C₂₀ unsubstituted or substituted aryl, C₆-C₂₀ unsubstituted or substituted aryl containing at least one heteroatom, C₇-C₂₀ unsubstituted or substituted alkaryl, or C₇-C₂₀ unsubstituted or substituted alkaryl containing at least one heteroatom; and
(iv) said at least one levulinic acid ester is optionally recovered.

2. The process of claim 1, wherein said basic catalyst is a metal catalyst selected from the group consisting of metal silicates, metal carbonates, metal bicarbonates, metal oxides, metal hydroxides, metal phosphates, metal aluminates, metal carboxylates and combinations thereof, said metal being selected from the group consisting of Group I and Group II elements of the Periodic Table.

3. The process of claim 1, wherein said basic catalyst is a homogeneous catalyst selected from the following groups: 1) trialkyl, tricycloalkyl, triaryl and trialkaryl amine, 2) trialkyl, tricycloalkyl, triaryl and trialkaryl diamine, 3) trialkyl, tricycloalkyl, triaryl and trialkaryl arsine, 4) trialkyl, tricycloalkyl, triaryl and trialkaryl diarsine, 5) trialkyl, tricycloalkyl, triaryl and trialkaryl phosphine, 6) trialkyl, tricycloalkyl, triaryl and trialkaryl diphosphine, 7) salts of groups 1-6, and 8) combinations of groups 1-7.

4. The process of claim 2, wherein said metal is selected from the group consisting of cesium, rubidium, lithium, sodium, potassium, strontium, scandium, calcium, barium, magnesium, compounds thereof and combinations thereof.

5. The process of claim 1 wherein said catalyst support is selected from the group consisting of carbon, alumina, silica, silica-alumina, silica-titania, silica-zirconia, titania, titania-alumina, zirconia, barium sulfate, calcium carbonate, strontium carbonate, compounds thereof and combinations thereof.

6. The process of claim 1, wherein said basic catalyst optionally comprises a catalyst promoter.

7. The process of claim 1 wherein the basic catalyst is supported, and the supported catalyst is selected from the group consisting of cesium on silica, cesium on titania, cesium on zirconia, cesium on silica-titania, cesium on silica-zirconia, rubidium on silica, rubidium on titania, rubidium on zirconia, rubidium on silica-titania, rubidium on silica-zirconia, lithium on silica, lithium on titania, lithium on zirconia, lithium on silica-titania, lithium on silica-zirconia, sodium on silica, sodium on titania, sodium on zirconia, sodium on silica-titania, sodium on silica-zirconia, potassium on silica, potassium on titania, potassium on zirconia, potassium on silica-titania, potassium on silica-zirconia, strontium on silica, strontium on titania, strontium on zirconia, strontium on silica-titania, strontium on silica-zirconia, scandium on silica, scandium on titania, scandium on zirconia, scandium on silica-titania, scandium on silica-zirconia, calcium on silica, calcium on titania, calcium on zirconia, calcium on silica-titania, calcium on silica-zirconia, barium on silica, barium on titania, barium on zirconia, barium on silica-titania, barium on silica-zirconia, magnesium on silica, magnesium on titania, magnesium on zirconia, magnesium on silica-titania and magnesium on silica-zirconia.

8. The process as recited in claim 2, wherein said metal catalyst is supported on a catalyst support and the content of the metal in the supported metal catalyst is from 0.1% to 40% by weight.

9. The process of claim 1 wherein the temperature of the reaction is from about 1° C. to about 300° C.

10. The process of claim 1 wherein the pressure of the reaction is from about 0.1 MPag to about 15 MPag.

11. The process of claim 1 wherein the content of the basic catalyst is from about 0.1% to about 50% by weight of the solution.

12. A composition comprising levulinic acid esters made by the process of claim 1.

13. The composition of claim 12 used as a fuel, an oxygenate for gasoline, an octane number-enhancing agent for gasoline, an oxygenate for diesel, a cetane number-enhancing agent for diesel or a fuel additive for biofuel.

14. A gasoline, diesel or biofuel comprising from 1% to 90% by volume of the composition of claim 12.

15. A gasoline, diesel or biofuel comprising from 1% to 50% by volume of the composition of claim 12.

16. A gasoline, diesel or biofuel comprising from 1% to 20% by volume of the composition of claim 12.

17. A process for manufacturing a fuel additive, the process comprising the process of claim 1.
